(19) United States
(12) Patent Application Publication
(10) Pub. No.: US 2020/0168337 A1
(43) Pub. Date: May 28, 2020

(54) TECHNIQUES TO ASSIST IN DIAGNOSIS AND TREATMENT OF INJURY AND ILLNESS

(71) Applicant: Vulcan Inc., Seattle, WA (US)

(72) Inventors: Richard Earl Simpkinson, Issaquah, WA (US); Keith Rosema, Seattle, WA (US); Omer Rosenbaum, Kirkland, WA (US); Richard Ignacio Zaragoza, Issaquah, WA (US); Michael Persiantsev, Shrewsbury, MA (US); Rusty Allen Gerard, Bothell, WA (US)

(21) Appl. No.: 16/696,981

(22) Filed: Nov. 26, 2019

Related U.S. Application Data
(60) Provisional application No. 62/771,560, filed on Nov. 26, 2018.
(60) Provisional application No. 62/797,144, filed on Jan. 25, 2019.
(60) Provisional application No. 62/822,738, filed on Mar. 22, 2019.

(51) Int. Cl. G16H 50/20; G16H 40/63; G16H 20/30; G16H 40/20; G16H 50/50; G16H 20/10; G16H 10/60; G06N 5/04; G06K 9/62; G06T 7/20; G06T 7/00

(52) U.S. Cl. CPC G16H 50/20; G16H 40/63; G16H 20/30; G16H 40/20; G16H 50/50; G16H 20/10; G06T 2207/30196; G06N 5/04; G06K 9/62; G06T 7/20; G06T 7/0012; G06T 2207/10016; G16H 10/60

(57) ABSTRACT

A diagnostic model, adapted to a condition of a patient, is maintained. A monitoring device obtains visual information of a patient. The visual information is converted to a format suitable as input to the diagnostic model. Output of the diagnostic model is used to generate a recommendation for at least one of diagnosis or treatment of the patient. A response to the diagnosis or treatment recommendation is generated.

### CROSS-REFERENCE TO RELATED APPLICATIONS

This application claims the benefit of U.S. Provisional Patent Application No. 62/771,560, filed Nov. 26, 2018, of U.S. Provisional Patent Application No. 62/797,144, filed Jan. 25, 2019, and of U.S. Provisional Patent Application No. 62/822,738, filed Mar. 22, 2019, the disclosures of which are herein incorporated by reference in their entirety.

### BACKGROUND

Augmented reality is a relatively new technology which involves incorporating computer-generated graphics into a view of a user's real physical environment. Typically, augmented reality applications are performed with devices such as smartphones or goggles, which incorporate a camera for capturing an image of the user's physical environment, and a display for presenting the augmented view.

Medical diagnosis and treatment typically begins with a visit to a doctor's office to receive a diagnosis and obtain a treatment plan. Following such visits, the patient is generally responsible for the execution of the treatment plan. However, for a variety of reasons, patients are often ill-equipped to do so. This is particularly true when the treatment of an injury or illness involves complex steps.

### BRIEF DESCRIPTION OF THE DRAWINGS

Various techniques will be described with reference to the drawings, in which:

FIG. 1 illustrates an example of a patient monitoring system, in accordance with an embodiment;

FIG. 2 illustrates an example of a patient monitoring and response system, in accordance with an embodiment;

FIG. 3 illustrates an example of a patient monitoring device, in accordance with an embodiment;

FIG. 4 illustrates an example process of monitoring and responding to participant health information, in accordance with an embodiment;

FIG. 5 illustrates an example of a customized patient health model, in accordance with an embodiment;

FIG. 6 illustrates an example of medical history information stored and present as a customized patient health model, in accordance with an embodiment;

FIG. 7 illustrates an example process of maintaining a customized patient model, in accordance with an embodiment;

FIG. 8 illustrates an example process of enhancing display of diagnostic information and associated four-dimensional image data;

FIG. 9 illustrates further aspects of enhancing display of diagnostic information and associated four-dimensional image data;

FIG. 10 illustrates an example process for improving patient physician feedback, in accordance with an embodiment; and

FIG. 11 illustrates an environment in which various embodiments can be implemented.

### DETAILED DESCRIPTION

Techniques and systems described herein relate to enhancing the diagnosis and treatment of injury and illness. In particular, techniques are disclosed for improving the diagnosis and treatment of illness, based on techniques which leverage various augmented reality techniques to enhance perception and improve collection of medically-relevant data. These techniques include those which facilitate the execution of a treatment plan and which leverage anatomical and physiological models to improve physician and patient understanding of an illness or injury and the treatment thereof.

In the preceding and following description, various techniques are described. For purposes of explanation, specific configurations and details are set forth in order to provide a thorough understanding of possible ways of implementing the techniques. However, it will also be apparent that the techniques described below may be practiced in different configurations without the specific details. Furthermore, well-known features may be omitted or simplified to avoid obscuring the techniques being described.

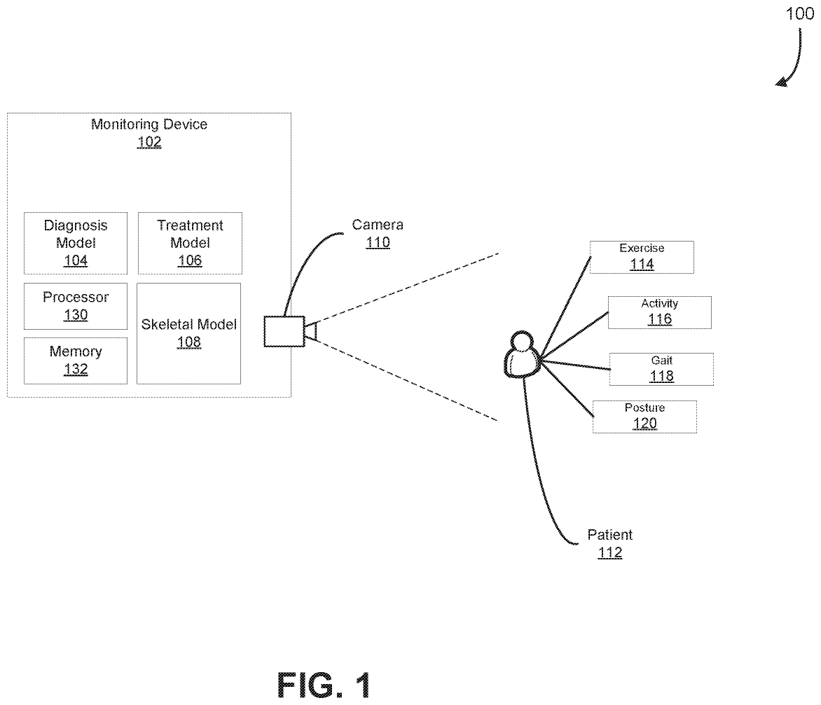

FIG. 1 illustrates an example 100 of a patient monitoring system, in accordance with an embodiment. A patient monitoring system, in at least one embodiment, collects information relevant to the diagnosis and treatment of an injury or illness, and provides information and assistance relative to the treatment of the injury or illness.

In at least one embodiment, a monitoring device 102 comprises a processor 130 and a memory 132. The processor 130 may include various forms of general-purpose or customized microprocessors capable of executing instructions stored on the memory 132. The memory 132 includes a non-transitory storage medium on which processor-executable instructions are stored. The monitoring device 102 may be a portable computing device, smartphone, internet-of-things device, and so on. The monitoring device 102 comprises a camera 110 or other sensor capable of collecting the information needed for performing the functions of the monitoring device 102. For example, rather than comprising the camera 110, the monitoring device might comprise LIDAR sensor(s), infrared sensors, audio microphones, and so on, individually or in combination.

The monitoring device 102 comprises instructions for processing data collected by the camera 110, or other similar devices, and maintaining a physiological model. In at least one embodiment, the monitoring device 102 comprises a skeletal model 108. The skeletal model 108 comprises data representing the position, pose, and posture of the patient 112. The skeletal model 108 may be represented using any suitable data structure, but (for example) may comprise data representing a “ball and stick” model of the patient 112. In embodiments, the skeletal model 108 includes representations of the patient's 112 joints, including information representing a direction and degree of extension that is possible for each joint. The information may further include indications of what is considered normal or abnormal direction or extension, what might be considered indicative of injury, and so forth.

The term model, as used herein, may refer to a computer-executable instructions for performing the functions attributed herein to the particular model. The model may further comprise data and subroutines, stored at least partially in a memory of a computing device, for simulating or gaining insight into data relevant to the model. For example, a skeletal model may comprise data and subroutines for simulating or predicting normal and abnormal movements. More generally, a diagnosis model might contain data and subroutines that assist in evaluating sensor data to determine when the sensor data is relevant to a medical diagnosis, and may further comprise data and subroutines for making a medical diagnosis. Likewise, a treatment model may contain data and subroutines for identifying sensor data relevant to medical treatment, and may further contain data and subroutines for providing assistance in providing medical treatment, e.g. by generating data that can be used to generate a display illustrating correct movement for a rehabilitative exercise. Further aspects of these and other models will be described herein.

By executing instructions stored in the memory 132, the processor 130 causes the monitoring device 102 to collect data for and maintain the skeletal model 108, such that the motion of the patient 112 is made available to the monitoring device 102 for further analysis. The skeletal model 108 includes representations of the patient's 112 position, pose, and posture over time. This “animated” skeletal model may therefore be used, by the monitoring device 102, to perform analysis of the patient's 112 exercise 114, activity 116, gait 118, or posture 120. In at least one embodiment, comparison of predicted movements by the skeletal model is compared to actual movement of the patient. The comparison may then be used to identify a potential injury, to suggest improvements in the execution of a rehabilitative exercise, and so forth.

By executing instructions stored in the memory 132, the processor 130 causes the monitoring device 102 to obtain, store, and apply data to a diagnosis model 104. The diagnosis model 104 may comprise information related to a condition applicable to the patient 112. The information includes data that indicates aspects of the patient's 112 exercise, activity, gait, posture, and so forth that may be relevant to the condition, and in particular includes information that may be used for analysis and interpretation of the skeletal model, or other data that may be collected by the monitoring device 102. For example, the diagnosis model 104 may indicate data which flags certain joints for observation and indicates that certain types of flexion or extension may be harmful, or indicative of an underlying problem. Similar techniques may be applied to other types of physiological models.

By executing instructions stored in the memory 132, the processor 130 causes the monitoring device 102 to obtain, store, and apply a treatment model 106. The information includes data that describes aspects of the patient's 112 treatment plan that may be evaluated based on the skeletal model 108, or based on other data collected by the monitoring device 102.

For example, a patient's treatment plan may require that certain exercises be performed. Prior to performing these exercises, the patient 112 may activate the monitoring device 102 and consent to its collection of video data. The monitoring device 102 uses the data to maintain the skeletal model 108 while the patient 112 performs the exercise. Using the treatment model 106, the monitoring device 102 evaluates the patient's performance of the exercise based on the treatment model 106, and provides feedback or other information. This can include, for example, indications that the patient is not performing the full range of movement required for the exercise to be effective, or that the patient may be performing the exercise in a way that might be harmful. The system may also track the patient's performance of the exercise, and indicate if additional repetitions of the exercise should be performed, or warn if the intended number of repetitions is being exceeded. The system may also utilize data represented by the diagnosis model 104 in order to provide this information.

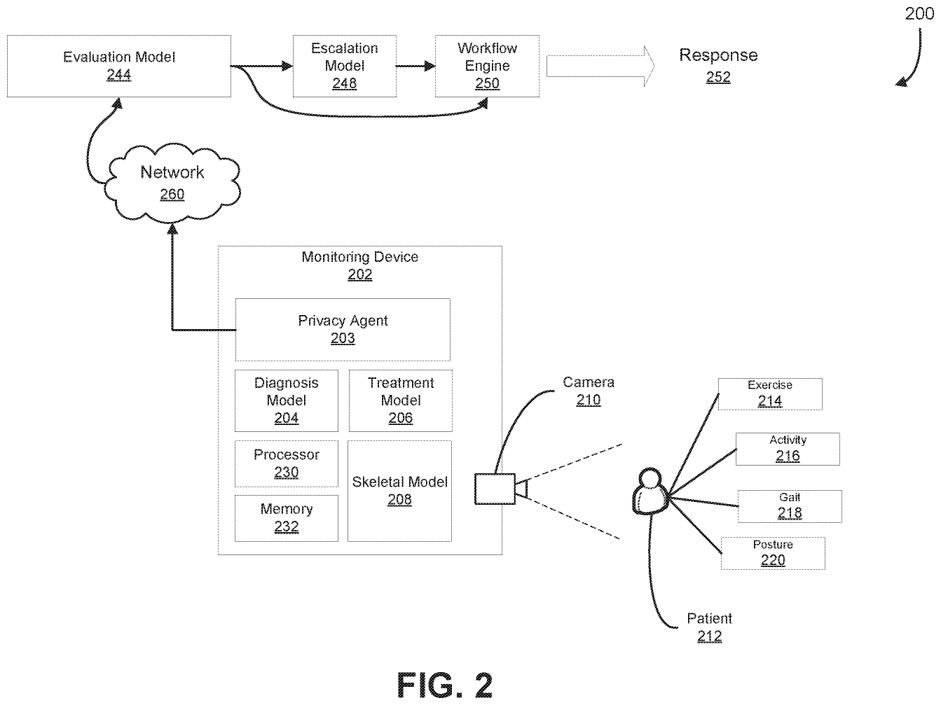

FIG. 2 illustrates an example of a patient monitoring and response system, in accordance with an embodiment. Elements of the example 200 illustrated in FIG. 2 may correspond to similar elements depicted in FIG. 1. Moreover, FIG. 2 depicts aspects of integrating the patient monitoring system of FIG. 1 with other systems and workflows, including those related to providing a response to a potential diagnosis.

In the example 200 of FIG. 2, data collected by the monitoring device 202 is shared, subject to parameters and authorization provided by the patient 212, with a cloud-based service that performs further analysis in order to generate a response 252 to a condition or event associated with the patient 212.

The monitoring device 202, which may correspond to the monitoring device 102 depicted in FIG. 1, may include a diagnosis model 204, treatment model 206, skeletal model 208, camera 210, processor 230, and memory 232 similar to those described with respect to FIG. 1. Likewise, monitoring device 202 may observe the exercise 214, activity 216, gait 218, and posture 220 of the patient 212 in a manner similar to that which is described regarding FIG. 1.

The monitoring device 202 further comprises a privacy agent 203. The privacy agent 203 facilitates the patient's 212 control over information that is shared, including but not necessarily limited to ensuring compliance to laws and regulations related to privacy.

In an embodiment, the privacy agent 203 uses the diagnosis model 204 and/or treatment model 206 to identify information that may be shared. For example, the patient 212 may consent to sharing information related to his exercise routine, but wish to prevent other information from being shared, or allow other information to be shared only if the data is anonymized. The monitoring device 202 can leverage the diagnosis model 204 or treatment model 206 to identify information related to the exercise routine, so that that information can be shared in accordance with the patient's wishes.

In another embodiment, medical personnel associated with the patient 212 can help identify information that is to be shared. For example, the patient 212 may wish to share all relevant medical information with his or her doctor, but might not know that certain forms of information—such as posture or gait information—might be considered relevant by the doctor. In embodiments, information configuring the diagnosis model 204 and treatment model 206 are sent from the doctor to the patient's monitoring device. These models 204, 206 are capable of identifying and collecting information related to the diagnosis and treatment, and sharing that information when appropriate, e.g., under the explicit or implicit authorization of the patient 212.

In the example 200 of FIG. 2, various additional services are hosted by a service provider, such as a cloud-based provider of computing services. These services may also be operated using proprietary data centers, or other facilities. The hosted services may comprise, in various embodiments, an evaluation model 244, an escalation model 248, and a workflow engine 250.

The evaluation model 244 is able to interpret data provided by the monitoring device 202 regarding the patient 212. In embodiments, the evaluation model 244 performs an evaluation of the data in order to cause an action to be taken by the workflow engine 250. Examples of such actions include scheduling follow-up action by a physician.

In cases and embodiments, the evaluation model 244 identifies exercises that have been performed as part of a rehabilitative treatment plan, and causes that information to be stored and provided to the patient's physical therapist or doctor. The evaluation model 244 may further identify deviations from the treatment plan, or potential causes for concern, and notify the patient's physical therapist or doctor.

In cases and embodiments, the evaluation model 244 identifies potential health conditions. For example, the evaluation model 244 may identify, based on data provided by the monitoring device 202, that the patient 212 has elevated blood pressure. The evaluation model 244 may then provide this information to the escalation model 248 and/or workflow engine 250 for further action.

Embodiments may comprise an escalation model 248 for determining when collected information suggests the need for further action. For example, regarding the patient's 212 elevated blood pressure, the escalation model 248 may determine to notify the patient's doctor and send a message to the patient 212, potentially including tips for lowering blood pressure. The evaluation model 244 and escalation model 248 may collectively identify conditions requiring immediate action, or assign a relative priority to observed conditions or collected information.

The workflow engine 250 tracks identified issues and ensures that an appropriate response is taken. For example, the identification of a patient's elevated blood pressure may suggest a coordinated response involving making sure the patient and the patient's doctor are notified, and recording the relevant information in the patient's medical record. The workflow engine 250 tracks the execution of these steps and ensures that each completes successfully.

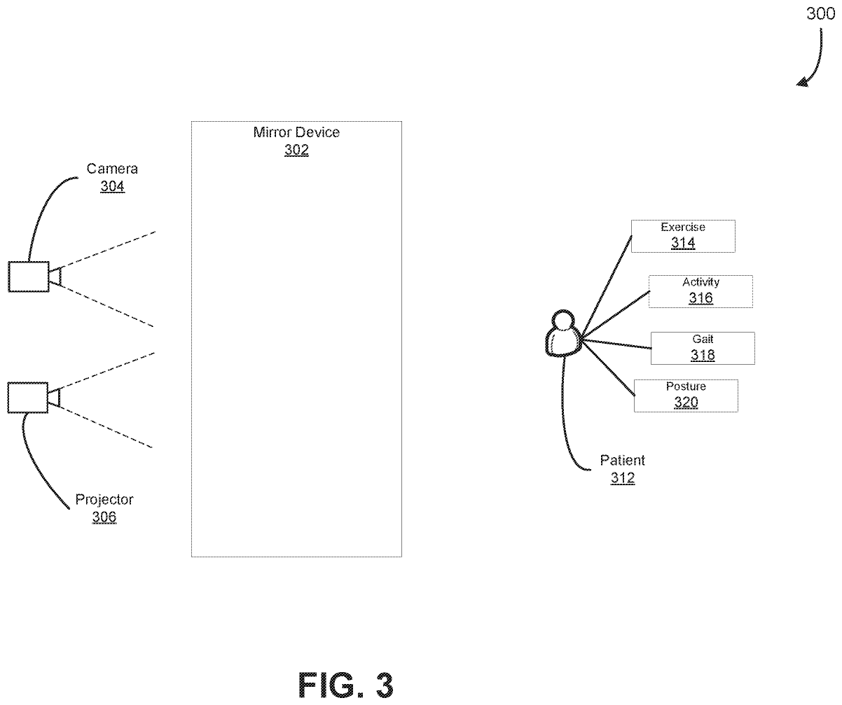

FIG. 3 illustrates an example of a patient monitoring device, in accordance with an embodiment. In the example 300 of FIG. 3, a monitoring device (such as those depicted in FIGS. 1 and 2) is implemented as a mirror 302 or other similar surface. The mirror device 302 may comprise a camera 304 and projector 306, and may further comprise the other components of a monitoring device as depicted in FIGS. 1 and 2. The camera 304 is configured to capture the patient's 312 image when the patient 312 looks in or is otherwise visible in the mirror. The projector 306 is capable of projecting images onto the surface of the mirror. Other devices may be substituted for the projector 306, such as a screen that is integrated into the mirror. In at least one embodiment, the mirror 302 is a computer display that functions as both a mirror and a display.

In an embodiment, the mirror device 302 displays images on the surface of the mirror 302 to provide information related to an exercise 314 or activity 316 being performed by the patient 312. The information may be provided while the exercise or activity is being performed. For example, while the patient 312 performs an exercise, the mirror device 302 may display augmented reality images suggesting an ideal path of movement for the exercise. The mirror device 302 might also display a count of the number of remaining repetitions, an indications of which muscle groups are affected by the exercise, and so forth. The mirror device might also monitor gait 318 and posture 320.

In an embodiment, the mirror device 302 monitors the patient's 312 intake of medicine. For example, patient may follow a routine in which the patient consumes medicine while in view of the mirror device 302. The mirror device 302 performs object identification on the medicine's container, and identifies the patient's consumption of the medicine. This may be done, for example, by identifying the medicine, identifying the step of opening the container, and identifying the act of consuming the medicine. The mirror device 302 may then display information indicating when the medicine was last taken.

Functions similar to those described in relation to the mirror device of FIG. 3 may also be performed on other monitoring devices, such as those depicted in FIGS. 1 and 2.

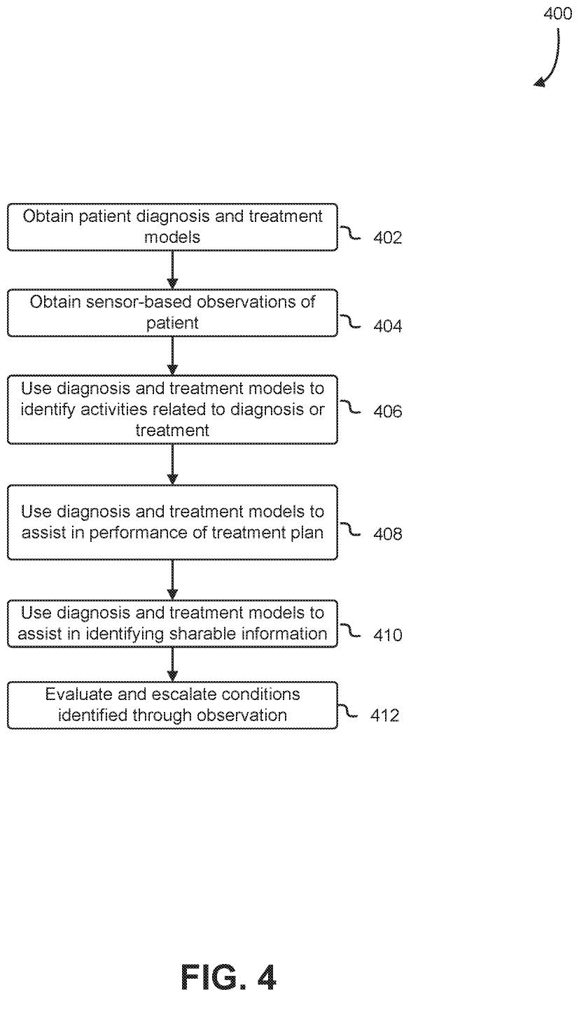

FIG. 4 illustrates an example process of monitoring and responding to participant health information, in accordance with an embodiment. Some or all of the process 400 (or any other processes described, or variations and/or combinations of those processes) may be performed under the control of one or more computer systems configured with executable instructions and/or other data, and may be implemented as executable instructions executing collectively on one or more processors. The executable instructions and/or other data may be stored on a non-transitory computer-readable storage medium (e.g., a computer program persistently stored on magnetic, optical, or flash media).

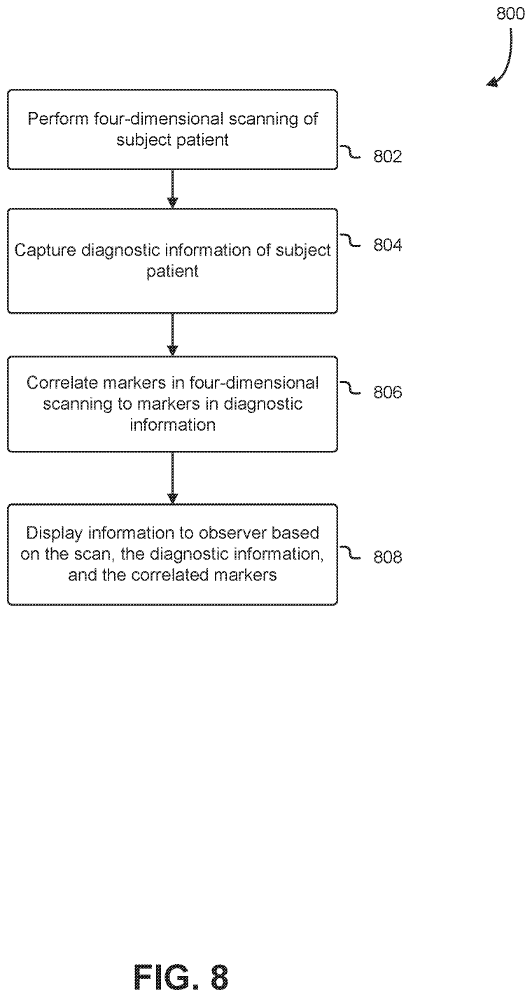

For example, some or all of process 400 may be performed by any suitable system comprising the monitoring device 102 depicted in FIG. 1, and/or other devices, such as a server in a data center, by various components of the environment 800 described in conjunction with FIG. 8, such as the one or more web servers 806 or the one or more application servers 808, by multiple computing devices in a distributed system of a computing resource service provider, or by any electronic client device such as the electronic client device 802.

At 402, the health monitoring system obtains diagnosis and treatment models for a patient. For example, this information may be downloaded to a monitoring device.

At 404, the system sensor-based observations of the patient. For example, a camera in a monitoring device may capture and store a visual record of the patient as the patient performs an exercise that is part of his or her treatment plan.

At 406, the system uses the diagnosis and treatment models to identify these activities. For example, the monitoring device may be made aware, based on the treatment models, of the exercises and individual is expected to perform, and uses this information to analyze the sensor data and determine that the patient is performing the exercise. Similar approaches may be applied to other treatment activities, such as the patient's ingestion of medicine.

At 408, the system uses the diagnosis and treatment models to assist in the performance of the treatment plan. For example, the model may be used to provide simulations of a correctly executed exercise, or to compare the patient's execution of the exercise with an idealized version.

At 410, the system uses the diagnosis and treatment models to assist in the identification of information that is related to the patient's condition, and share that information with the patient's doctor or other medical personnel, if authorized by the patient to do so.

At 412, the system evaluates and escalates conditions (or other events, data, and so forth) that warrant escalation and are permitted to be shared. For example, the system may obtain various observations regarding the patient's health, such as temperature, heart rate, blood pressure, and so forth. Based on this information, a diagnostic model may identify a potentially dangerous condition, and trigger a workflow in which a response to the potentially dangerous condition is escalated to appropriate individuals, such as the patient's doctor.

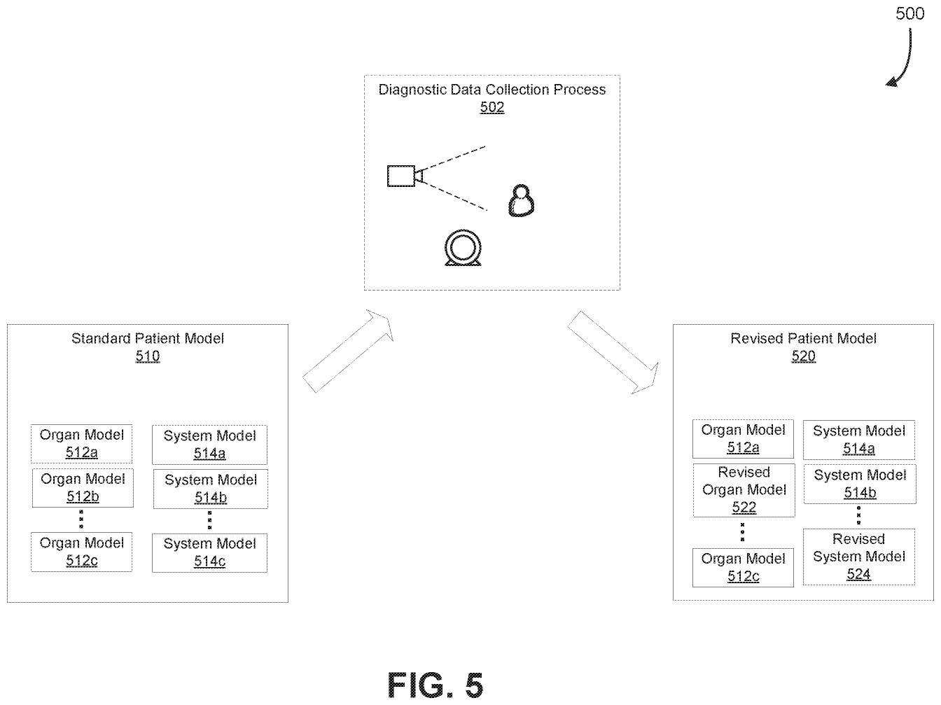

FIG. 5 illustrates an example of a customized patient health model, in accordance with an embodiment. In the example 500 of FIG. 5, a standard patient model 510 comprises data and subroutines that simulate various aspects of a “standard” patient. Here, “standard” refers to a baseline representation of the simulated patient. For example, one standard patient model 510 might simulate a human subject at 50 years of age, while another might simulate a human subject at 25 years of age. In other cases and embodiments, a standard patient model might represent another species, such as a dog or cat, which is being monitored for diagnosis or treatment. Other embodiments might represent baseline versions of a subject with a specific condition, such as a patient with cancer or Parkinson's disease.

The standard patient model 510 may comprise various sub-components, including organ models 512a-c and system models 514a-c. The organ models 512a-c, in embodiments, may be used to predict aspects of the organ's operation, such as the liver's production of bile, the heart's pumping function, and so forth. In other embodiments, physical structure is represented, such as the size and position of the organ. Each of these models may be useful in a different context.

The system models 514a-c may represent the functioning and/or physical structure of various anatomical or physiological systems, such as the lymphatic or circulator systems.

The standard patient model 510 may also comprise a model of structure features, such as the typical size and position of various anatomical components.

A diagnostic data collection process 502 comprises, in various embodiments, a multi-faceted approach to collecting diagnostic data. Data from a variety of sources, such as computerized tomography, magnetic resonance imaging, x-rays, genetic testing, and so forth may be collected. In various embodiments, data collecting from monitoring systems and devices, such as those depicted in FIGS. 1-3, may also be collected.

Data collected through the data collection process 502 is used to generate a revised patient model 520. For example, if an ultrasound test is performed on the liver, a revised organ model 522 may be generated to conform to the findings. For example, the revised organ model 522 might represent the fact that the liver of the specific patient is enlarged. This, in turn, may cause other organ models to be revised. For example, the enlarged liver might cause another organ to be displaced. Similarly, a decline in liver function might be known to impact the functioning of other organs. These effects can be modelled either by directly updating the model of the affected organ, or by simulating the effect based on the revised model 522 for the liver.

As additional data is collected, the revised patient model 520 can be further revised. In some cases and embodiments, multi-faceted data can be used to improve the model. For example, a computerized tomography scan may obtain different information than a magnetic resonance imaging scan of the same organ. Data for both of these scans, as well as any other available information concerning the organ, can be applied to the model to form a more accurate representation of the organ. Similar modifications may be made to the various system models, e.g., by updating the revised system model 524 based on blood test results.

In at least one embodiment, the various models just described are integrated into a system, such as the one depicted in FIG. 2, for diagnosis and treatment of injury. For example, the various models just described may process information obtained through a monitoring device 202, and generate data that can in turn be analyzed to diagnose and injury or illness, or to assess a patient's progress in treating an injury or illness. For example, the various models just described, individually or in various combinations, may generate data that is predictive of a patient's condition at various times during a course of treatment.

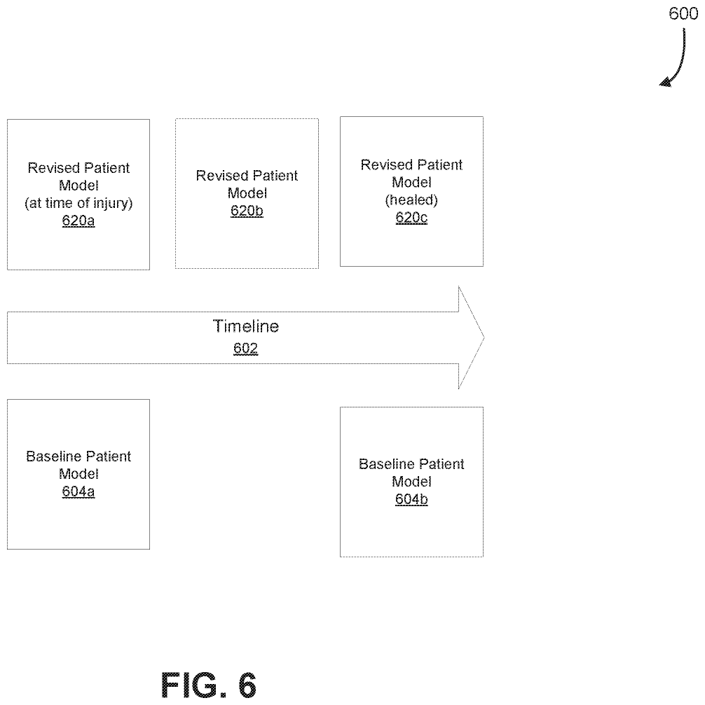

FIG. 6 illustrates an example of medical history information stored and present as a customized patient health model, in accordance with an embodiment. As depicted in the example 600, a revised patient model 620a may evolve over a timeline 602 based on the acquisition of new data.

The revised patient model 620, in various embodiments, is usable as a medical record. In particular, the revised patient model 620 can be used to provide insight into a patient's health as of a given time, and as a means of understanding changes to the patient's health over time.

For example, a snapshot of the patient's model 620a may be compared to a baseline patient model 604a,b at the time of an injury. Subsequent snapshots 620b may be used to examine the patient's progress, and a snapshot 320c at a later time, when the patient is considered healed, can be compared to another baseline 604b of a standard patient. This approach thus facilitates comparisons between the patient's health at various times, as well as comparisons with “standard” patients.

Note that rather than merely listing changes to the patient's health, or merely rendering those changes graphically, the revised patient model 620a permits evaluation and exploration of the patient's functioning as of a particular time. For example, if a seemingly new condition is discovered in the patient, the doctor might use the model 620 to go back in time to see if the condition might likely have existed at an earlier time, based on the predictions of the earlier model.

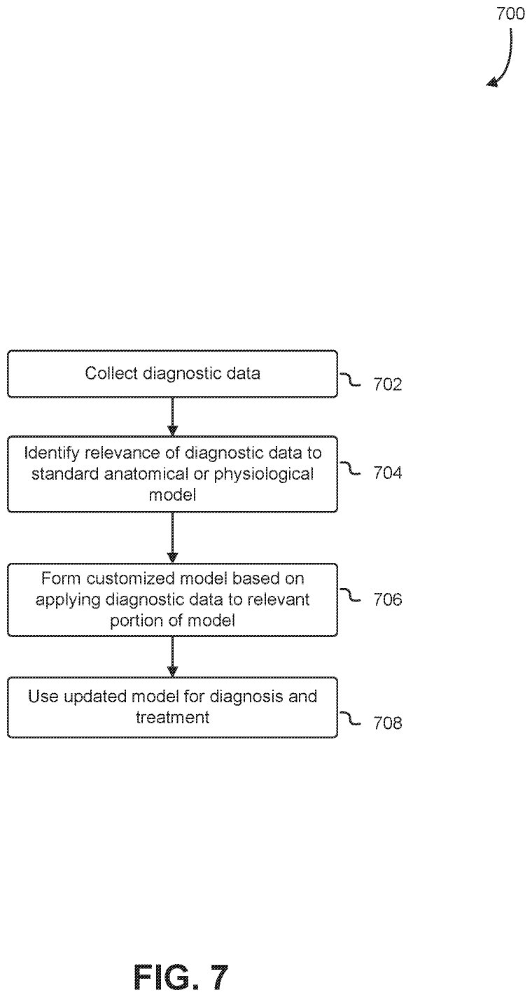

FIG. 7 illustrates an example process of maintaining a customized patient model, in accordance with an embodiment. Some or all of the process 700 (or any other processes described, or variations and/or combinations of those processes) may be performed under the control of one or more computer systems configured with executable instructions and/or other data, and may be implemented as executable instructions executing collectively on one or more processors. The executable instructions and/or other data may be stored on a non-transitory computer-readable storage medium (e.g., a computer program persistently stored on magnetic, optical, or flash media).

For example, some or all of process 700 may be performed by any suitable system, such as by various components of the environment 800 described in conjunction with FIG. 8, such as the one or more web servers 806 or the one or more application servers 808, by multiple computing devices in a distributed system of a computing resource service provider, or by any electronic client device such as the electronic client device 802.

At 702, the system collects or otherwise obtains diagnostic data. At 704, the relevance of the acquired data to a standardized anatomical or physiological model is identified. For example, the system may determine which organ(s) are implicated by recently acquired diagnostic data.

At 706, the system forms a customized model by applying the diagnostic data to the relevant portions of the standard model. At 708, the updated model is used for diagnosis and treatment.

In an embodiment, diagnostic data comprising a three-dimensional scan of an organ or other anatomical component is obtained. The relevant portion(s) of the standard anatomical model is then updated, based on the three-dimensional scan. The updated model (which may be described as a customized model) includes a more accurate representation of the size, position, and structure of the relevant organ(s). In addition, any other portions of the model that are implicated by the new data can also be adjusted. This model can then be used for diagnosis and treatment. In one example, an anatomic model is adjusted to represent a particular patient's anatomy. The model is then used to simulate the effect of different operating positions on a potential surgery, based on the more accurate representation of the patient's anatomy.

FIG. 8 illustrates an example process of enhancing display of diagnostic information and associated four-dimensional image data. Some or all of the process 800 (or any other processes described, or variations and/or combinations of those processes) may be performed under the control of one or more computer systems configured with executable instructions and/or other data, and may be implemented as executable instructions executing collectively on one or more processors. The executable instructions and/or other data may be stored on a non-transitory computer-readable storage medium (e.g., a computer program persistently stored on magnetic, optical, or flash media).

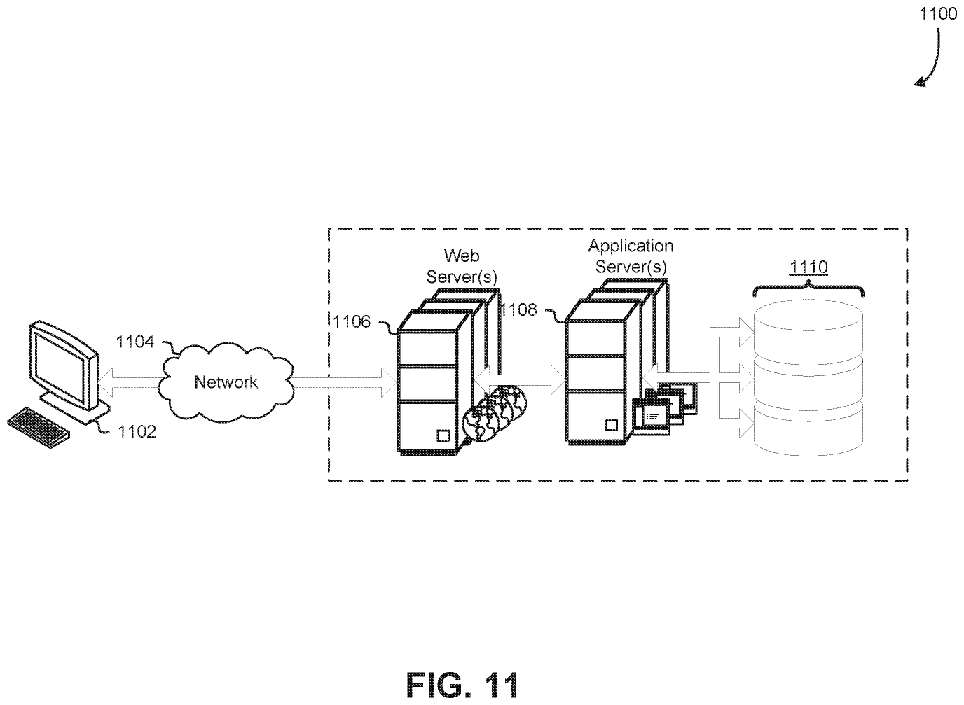

For example, some or all of process 800 may be performed by any suitable system comprising an application server and/or other devices, such as a server in a data center, by various components of the environment 1000 described in conjunction with FIG. 11, such as the one or more web servers 1106 or the one or more application servers 1108, by multiple computing devices in a distributed system of a computing resource service provider, or by any electronic client device such as the electronic client device 1102.

At 802, a four-dimensional scan is made of a subject patient. Here, four-dimensional refers to the collection of three-dimensional image information over time. For example, a three-dimensional scan might be made of a patient's arm, as the patients arm moves throughout a range of motion.

At 804, diagnostic information is collected from the subject patient. For example, x-ray, magnetic resonance imaging (“Mill”), ultrasound, or other diagnostic information may be collected. The information may further be pertinent to the same body part that was scanned by the four-dimensional scanner.

At 806, markers in the scan are correlated to markers in the diagnostic information. Various forms of marker information may be included in both the four-dimensional scan and the diagnostic information. For example, the four-dimensional scan might produce information which includes markers identifying reference points on the patient's anatomy. The diagnostic information might include similar information, corresponding for example to the same reference points. Using a patient's arm as an example, both the scan and a sequence of MRI information might include markers to indicate reference points, such as the patient's elbow and wrist joints.

At 808, information is displayed based to an observer based on the scan, the diagnostic information, and the correlated markers. In an example, a display is rendered to show the patient's arm as it moves throughout the range of motion, onto which data from the MM is superimposed. For example, if the patient suffers from “tennis elbow,” the patient's arm might be rendered such that the nature of the injury is made more apparent or understandable using portions of the MM data projected onto the moving image of the patient's arm.

In an embodiment, the aforementioned techniques are used to enhance training of medical personnel.

In an embodiment, the aforementioned techniques are used to enhance patient understanding of an injury and approaches to treatment.

In an embodiment, the aforementioned techniques are used to enhance a doctor's ability to diagnose and/or treat an injury.

For example, a diagnostic model, such as a skeletal model, may be linked to diagnostic information via the use of correlated markers. The diagnostic information may contain information obtained from a variety of sources. As a rehabilitative exercise is performed, a range of patient motion is observed. The diagnostic model may be used in this instance to provide information to enhance awareness of various issues, such as to illustrate prior results of a range of motion test, to highlight past injuries involving bone or muscle groups involved in the rehabilitative test, to highlight goals for the rehabilitative exercise, and so forth. These capabilities may be further enhanced by the ready availability of correlated diagnostic information.

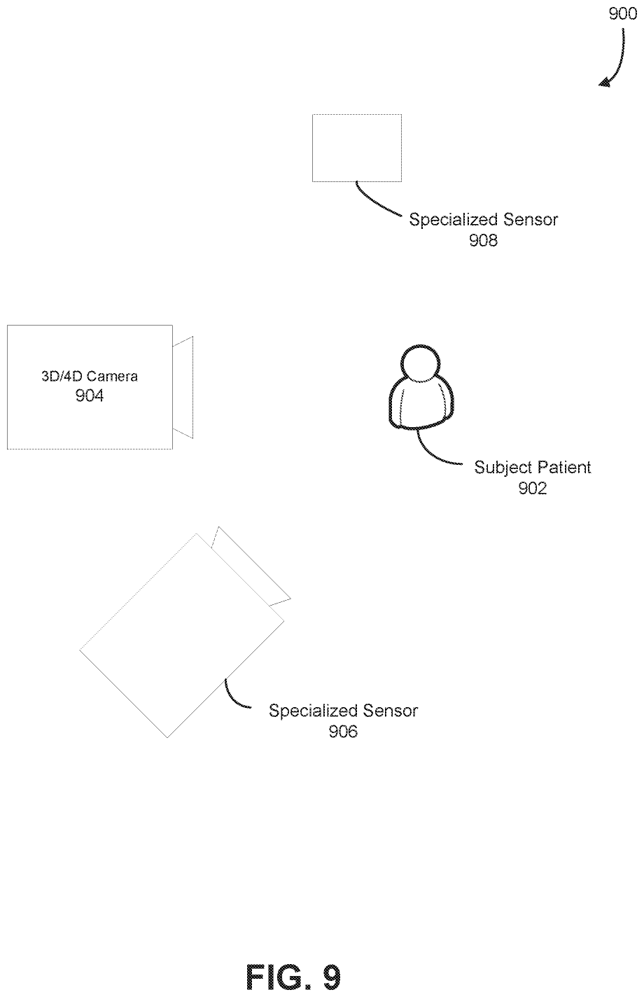

FIG. 9 illustrates further aspects of enhancing display of diagnostic information and associated four-dimensional image data. In the example 900 of FIG. 9, information concerning subject patient 902 is collected from a variety of sensor types.

A 3D/4D camera 904 captures three-dimensional images, over time, of the subject patient 902. Here, the fourth dimension refers generally to time, so that image data, including three-dimensional diagnostic data as well as visual data, can be observed over time.

Other sensors may collect additional information to make up for deficiencies of the 3D/4D camera 904. For example, a specialized sensor 904 might capture high-resolution images of the subject patient 902 to make up for resolution deficiencies of the 3D/4D camera 904. Similarly, another specialized sensor 906 might collect audio information (e.g., the sounds of a heart beating or the sounds of blood flowing through a blood vessel), ultrasound data, x-ray data, and so forth.

The output of the various cameras and sensors 904-908 may be correlated by various markers. For example, the output may be tagged with time information, which might allow for audio information to be correlated to visual information. Likewise, diagnostic information might also be tagged with data indicating a position on the body of the subject patient 902, to allow for diagnostic images to be correlated.

In at least one embodiment, a diagnostic model, such as a skeletal model, is used to correlate diagnostic information collected from various sources. For example, an x-ray obtained of an abdominal portion of a patient might be tagged to a corresponding abdominal portion of a three-dimensional skeletal model. The same might be done for magnetic resonance imaging (“Mill”) data. By correlating the data to a common point on a skeletal model, diagnostic data relevant to a common point on the body can be easily cross-correlated, even when a patient is in motion. The skeletal model may be used, for example, to provide an animated view of diagnostic information collected from multiple sources. For example, an animated view of a patient may be generated, as the patient engages in a range of motion. The animated view may include “close ups” of an involved body part, e.g. a close-up of the body part obtained via x-ray and a close-up of the body part obtained via MM.

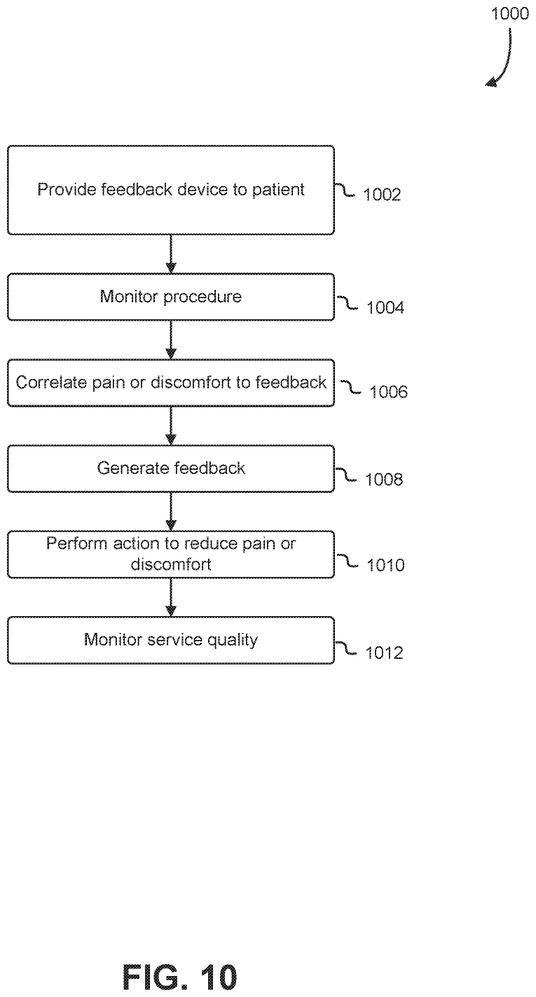

FIG. 10 illustrates an example process for improving patient physician feedback, in accordance with an embodiment. Although FIG. 10 is illustrated as a sequence of operations, it should not be construed in a manner which would limit the scope of the present disclosure to only those embodiments that conform to the depicted sequence. For example, in various embodiments, the depicted operations may be reordered or performed in parallel, except where the depicted sequence is logically required, such as when the output of one step is the input to a subsequent step.

The element 1002 depicts providing a feedback device to a patient. For example, a feedback device may comprise a compressible back, compressible trigger, or other device capable of measuring a degree of pressure applied to it. The patient may be given instructions to grip the device more tightly when pain or discomfort is encountered, such that more intense discomfort is indicated with a tighter grip on the device. This reaction may be natural to many patients.

At 1004, a medical or dental procedure is monitored in order to observe activity and provide feedback and correlation between the observed activity and the indications of pain or discomfort being indicated by the patient.

In an embodiment, the procedure is monitored by video camera. The video is processed by a machine learning network in order to identify correlations between the procedure and an indication of pain or discomfort.

In an embodiment, the procedure is monitored by audio. For example, the system may learn to correlate indications by the physician (e.g., “we are now perform step X in the procedure”) to indications of pain or discomfort by the physician.

In an embodiment, activity of associated instruments, such as a dentist's drill, is correlated to the indications provided by the patient. These various correlation activities are depicted by element 1006. In at least one embodiment, the correlation is facilitated by one or more diagnostic models. In at least one embodiment, the correlation is facilitated by a model of the procedure, which can be kept up-to-date by a recording video and/or audio data of the procedure, analyzing the data to convert it to a format suitable for input to the model, and using the input to update the model. Here, updates to the model may include updates which cause the state of the model to reflect a corresponding stage of the procedure.

At 1008, feedback is generated to the patient and/or physician.

In an embodiment, the physician is provided immediate feedback to indicate that the patient is in discomfort. For example, the system may use visual cues such as a change in lighting, or an audio cue, to indicate that a recent activity has been reported as being painful or discomforting.

In an embodiment, the physician is provided with an indication of what activity caused the discomfort. For example, video monitoring of a dental procedure might permit correlation of activity on a particular tooth with pain. The system may therefore provide an indication, to the physician, regarding that tooth.

In an embodiment, the system performs and action, as depicted by 1010, to reduce the pain or discomfort. For example, the system might react to an indication of pain or discomfort by playing soft music, providing an alternative stimulus to counter the pain, and so forth.

In an embodiment, the patient is provided with information regarding the procedure. For example, based on the monitoring, the system may provide an indication of what steps are remaining in the procedure, and how long the current step of the procedure is likely to take.

In an embodiment, the system provides the patient or physician with information regarding what portions of the procedure are typically found to be most painful. For example, prior monitoring of the procedure, for both a wide variety of patients and for the particular patient, can be used to derive information regarding which portions of the procedure are likely to be painful.

Element 1012 depicts monitoring service quality. For example, the system may compare indications of pain or discomfort experienced during the procedure to other examples of similar procedures. By doing so, the system can provide an indication of the quality of service that was provided. In some embodiments, other quality metrics can also be obtained. For example, the physician's bedside manner might be monitored, with suitable protections to ensure privacy of the patient and physician, in order to provide an indication of whether the procedure met various quality goals.

In an example embodiment, a system comprises at least one processor and a memory comprising instructions that, in response to execution by the at least one processor, cause the system to at least maintain a diagnostic model of a patient, the diagnostic model adapted based at least in part on a condition of the patient; receive information usable as input to the diagnostic model, the information obtained from a monitoring device located in an environment of the patient; and generate a recommendation of at least one of diagnosis or treatment, the recommendation based at least in part on output of the diagnostic model, the output based at least in part on the input obtained from the monitoring device.

In a further aspect of the example embodiment, the information received from the monitoring device is screened, based at least in part on a second diagnostic model, for relevance to at least one of diagnosis or treatment.

In a further aspect of the example embodiment, the memory comprises instructions that, in response to execution by the at least one processor, cause the system to at least track movement of the patient observed by the monitoring device; compare the tracked movement to movements predicted based at least in part on a skeletal model and information indicative of a rehabilitative exercise; and generate the recommendation based at least in part on the comparison.

In a further aspect of the example embodiment, the memory comprises instructions that, in response to execution by the at least one processor, cause the system to at least receive information, obtained by the monitoring device, indicative of compliance with a treatment regime, wherein sharing of the information by the monitoring device is authorized by the patient.

In a further aspect of the example embodiment, the recommendation comprises information indicative of a diagnosis of at least one of an injury or illness.

In a further aspect of the example embodiment, the memory comprises instructions that, in response to execution by the at least one processor, cause the system to at least initiate a workflow that, upon execution, schedules a follow-up action in response to the diagnosis.

In an example embodiment of a computer-implemented method, the method comprises maintaining a diagnostic model of a patient, the diagnostic model adapted based at least in part on a condition of the patient; receiving information usable as input to the diagnostic model, the information obtained from a monitoring device located in an environment of the patient; and generating a recommendation of at least one of diagnosis or treatment, the recommendation based at least in part on output of the diagnostic model, the output based at least in part on the input obtained from the monitoring device.

In a further aspect of the example embodiment, the condition to which the diagnostic model is adapted comprises at least one of physiological data or anatomical data particular to the patient.

In a further aspect of the example embodiment, the diagnostic model simulates a physical or anatomical function.

In a further aspect of the example embodiment, the computer-implemented method further comprises screening information collected from a monitoring device for authorization to use the information, based at least in part on a second diagnostic model. In a further aspect of the example embodiment, the second diagnostic model determines relevance of the information to a medical condition for which monitoring is authorized by a user of the monitoring device.

In a further aspect of the example embodiment, the recommendation comprises information indicative of performance of a rehabilitative exercise. In a further aspect, the computer-implemented method also comprises tracking movement of the patient observed by the monitoring device; comparing the tracked movement to movements predicted based at least in part on a skeletal model and information indicative of a rehabilitative exercise; and generating the recommendation based at least in part on the comparing.

In a further aspect of the example embodiment, the computer-implemented method further comprises providing a reminder to assist in compliance with a treatment regime, based at least in part on information indicative of compliance received from the monitoring device, wherein sharing of the information by the monitoring device is authorized by the patient.

In a further aspect of the example embodiment, the computer-implemented method further comprises obtaining the information indicative of compliance by recognizing, in video data obtained from the monitoring device, an image of a container of medication.

In an example embodiment of a computer program product, a non-transitory computer-readable storage medium having stored thereon instructions that, in response to execution by at least one processor of a computer, cause the computer to at least maintain a diagnostic model of a patient; receive visual information obtained by a camera of a monitoring device; convert the visual information to a format usable as input to the diagnostic model; and obtain a recommendation of at least one of diagnosis or treatment, the recommendation based at least in part on output of the diagnostic model.

In a further aspect of the example embodiment, conversion of the visual information to the format usable as input to the diagnostic model is based at least in part on a skeletal model of the patient.

In a further aspect of the example embodiment, the recommendation comprises instructions for performing a rehabilitative exercise, the instructions displayed in a visual format by the monitoring device.

In a further aspect of the example embodiment, the non-transitory computer-readable storage medium has stored thereon instructions that, in response to execution by at least one processor of a computer, cause the computer to at least detect, in visual information obtained by the monitoring device, information indicative of compliance with a medication regime, the visual information authorized for use in detecting compliance; and generate the recommendation based at least in part on the information indicative of compliance with the medication regime.

In a further aspect of the example embodiment, the non-transitory computer-readable storage medium has stored thereon instructions that, in response to execution by at least one processor of a computer, cause the computer to at least track movement of the patient observed by the monitoring device; compare the tracked movement to movements predicted based at least in part on a skeletal model and information indicative of a rehabilitative exercise; and generate the recommendation based at least in part on the comparison.

In an example embodiment, a method of performing a medical or rehabilitative treatment comprises providing a patient with a feedback device while performing the treatment, and correlating input from the feedback device to a model of the treatment. The model of the treatment is kept up-to-date by observation of the treatment via a video collection device and processing by other associated models. Feedback from the device provides indications of pain or discomfort that may occur during the treatment. Correlation of the feedback with the up-to-date model permits identification of particular problem points in the procedure.

FIG. 11 illustrates aspects of an example environment 1100 for implementing aspects in accordance with various embodiments. As will be appreciated, although a web-based environment is used for purposes of explanation, different environments may be used, as appropriate, to implement various embodiments. The environment includes an electronic client device 1102, which can include any appropriate device operable to send and/or receive requests, messages, or information over an appropriate network 1104 and convey information back to a user of the device. Examples of such client devices include personal computers, cell phones, handheld messaging devices, laptop computers, tablet computers, set-top boxes, personal data assistants, embedded computer systems, electronic book readers, and the like.

The environment 1100 in one embodiment is a distributed and/or virtual computing environment utilizing several computer systems and components that are interconnected via communication links, using one or more computer networks or direct connections. However, it will be appreciated by those of ordinary skill in the art that such a system could operate equally well in a system having fewer or a greater number of components than those illustrated in FIG. 11. Thus, the depiction in FIG. 11 should be taken as being illustrative in nature and not limiting to the scope of the disclosure.

The network 1104 can include any appropriate network, including an intranet, the Internet, a cellular network, a local area network, a satellite network or any other network, and/or combination thereof. Components used for such a system can depend at least in part upon the type of network and/or environment selected. Many protocols and components for communicating via such network 1104 are well known and will not be discussed in detail. Communication over the network 1104 can be enabled by wired or wireless connections and combinations thereof. In an embodiment, the network 1104 includes the Internet and/or other publicly-addressable communications network, as the environment 1100 includes one or more web servers 1106 for receiving requests and serving content in response thereto, although for other networks an alternative device serving a similar purpose could be used as would be apparent to one of ordinary skill in the art.

The illustrative environment 1100 includes one or more application servers 1108 and data storage 1110. It should be understood that there can be several application servers, layers or other elements, processes or components, which may be chained or otherwise configured, which can interact to perform tasks such as obtaining data from an appropriate data store. Servers, as used, may be implemented in various ways, such as hardware devices or virtual computer systems. In some contexts, “servers” may refer to a programming module being executed on a computer system. As used, unless otherwise stated or clear from context, the term “data store” or “data storage” refers to any device or combination of devices capable of storing, accessing, and retrieving data, which may include any combination and number of data servers, databases, data storage devices, and data storage media, in any standard, distributed, virtual, or clustered environment.

The one or more application servers 1108 can include any appropriate hardware, software and firmware for integrating with the data storage 1110 as needed to execute aspects of one or more applications for the electronic client device 1102, handling some or all of the data access and business logic for an application. The one or more application servers 1108 may provide access control services in cooperation with the data storage 1110 and is able to generate content including, text, graphics, audio, video, and/or other content usable to be provided to the user, which may be served to the user by the one or more web servers 1106 in the form of HyperText Markup Language (HTML), Extensible Markup Language (XML), JavaScript, Cascading Style Sheets (CS S), JavaScript Object Notation (JSON), and/or another appropriate client-side structured language. Content transferred to the electronic client device 1102 may be processed by the electronic client device 1102 to provide the content in one or more forms including forms that are perceptible to the user audibly, visually, and/or through other senses. The handling of all requests and responses, as well as the delivery of content between the electronic client device 1102 and the one or more application servers 1108, can be handled by the one or more web servers 1106 using PHP: Hypertext Preprocessor (PHP), Python, Ruby, Perl, Java, HTML, XML, JSON, and/or another appropriate server-side structured language in this example. Further, operations described as being performed by a single device may, unless otherwise clear from context, be performed collectively by multiple devices, which may form a distributed and/or virtual system.

Each server typically will include an operating system that provides executable program instructions for the general administration and operation of that server and typically will include a computer-readable storage medium (e.g., a hard disk, random access memory, read only memory, etc.) storing instructions that, when executed (i.e., as a result of being executed) by a processor of the server, allow the server to perform its intended functions.

The data storage 1110 can include several separate data tables, databases, data documents, dynamic data storage schemes, and/or other data storage mechanisms and media for storing data relating to a particular aspect of the present disclosure. For example, the data storage 1110 may include mechanisms for storing various types of data and user information, which can be used to serve content to the electronic client device 1102. The data storage 1110 also is shown to include a mechanism for storing log data, such as application logs, system logs, access logs, and/or various other event logs, which can be used for reporting, analysis, or other purposes. It should be understood that there can be many other aspects that may need to be stored in the data storage 1110, such as page image information and access rights information, which can be stored in any of the above listed mechanisms as appropriate or in additional mechanisms in the data storage 1110. The data storage 1110 is operable, through logic associated therewith, to receive instructions from the one or more application servers 1108 and obtain, update, or otherwise process data in response thereto. The one or more application servers 1108 may provide static, dynamic, or a combination of static and dynamic data in response to the received instructions. Dynamic data, such as data used in web logs (blogs), shopping applications, news services, and other applications may be generated by server-side structured languages as described or may be provided by a content management system (CMS) operating on, or under the control of, the one or more application servers 1108.

In one embodiment, a user, through a device operated by the user, can submit a search request for a match to a particular search term. In this embodiment, the data storage 1110 might access the user information to verify the identity of the user and obtain information about items of that type. The information then can be returned to the user, such as in a results listing on a web page that the user is able to view via a browser on the electronic client device 1102. Information related to the particular search term can be viewed in a dedicated page or window of the browser. It should be noted, however, that embodiments of the present disclosure are not necessarily limited to the context of web pages, but may be more generally applicable to processing requests in general, where the requests are not necessarily requests for content.

The various embodiments further can be implemented in a wide variety of operating environments, which in some embodiments can include one or more user computers, computing devices, or processing devices that can be used to operate any of a number of applications. User or client devices can include any of a number of computers, such as desktop, laptop, or tablet computers running a standard operating system, as well as cellular, wireless, and handheld devices running mobile software and capable of supporting a number of networking and messaging protocols. Such a system also can include a number of workstations running any of a variety of commercially available operating systems and other known applications for purposes such as development and database management. These devices also can include other electronic devices, such as dummy terminals, thin-clients, gaming systems, and other devices capable of communicating via the network 1104. These devices also can include virtual devices such as virtual machines, hypervisors, and other virtual devices capable of communicating via the network 1104.

Various embodiments of the present disclosure utilize the network 1104 that would be familiar to those skilled in the art for supporting communications using any of a variety of commercially available protocols, such as Transmission Control Protocol/Internet Protocol (TCP/IP), User Datagram Protocol (UDP), protocols operating in various layers of the Open System Interconnection (OSI) model, File Transfer Protocol (FTP), Universal Plug and Play (UpnP), Network File System (NFS), and Common Internet File System (CIFS). The network 1104 can be, for example, a local area network, a wide-area network, a virtual private network, the Internet, an intranet, an extranet, a public switched telephone network, an infrared network, a wireless network, a satellite network, and any combination thereof. In some embodiments, connection-oriented protocols may be used to communicate between network endpoints. Connection-oriented protocols (sometimes called connection-based protocols) are capable of transmitting data in an ordered stream. Connection-oriented protocols can be reliable or unreliable. For example, the TCP protocol is a reliable connection-oriented protocol. Asynchronous Transfer Mode (ATM) and Frame Relay are unreliable connection-oriented protocols. Connection-oriented protocols are in contrast to packet-oriented protocols such as UDP that transmit packets without a guaranteed ordering.

In embodiments utilizing the one or more web servers 1106, the one or more web servers 1106 can run any of a variety of server or mid-tier applications, including Hypertext Transfer Protocol (HTTP) servers, FTP servers, Common Gateway Interface (CGI) servers, data servers, Java servers, Apache servers, and business application servers. The server(s) also may be capable of executing programs or scripts in response to requests from user devices, such as by executing one or more web applications that may be implemented as one or more scripts or programs written in any programming language, such as Java®, C, C# or C++, or any scripting language, such as Ruby, PHP, Perl, Python, or TCL, as well as combinations thereof. The server(s) may also include database servers, including those commercially available from Oracle®, Microsoft®, Sybase®, and IBM® as well as open-source servers such as MySQL, Postgres, SQLite, MongoDB, and any other server capable of storing, retrieving, and accessing structured or unstructured data. Database servers may include table-based servers, document-based servers, unstructured servers, relational servers, non-relational servers, or combinations of these and/or other database servers.

The environment 1100 can include a variety of data stores and other memory and storage media as discussed above. These can reside in a variety of locations, such as on a storage medium local to (and/or resident in) one or more of the computers or remote from any or all of the computers across the network 1104. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, a central processing unit (CPU or processor), an input device (e.g., a mouse, keyboard, controller, touch screen, or keypad), and an output device (e.g., a display device, printer, or speaker). Such a system may also include one or more storage devices, such as disk drives, optical storage devices, and solid-state storage devices such as random access memory (RAM) or read-only memory (ROM), as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within a working memory device, including an operating system and application programs, such as a client application or web browser. In addition, customized hardware might also be used and/or particular elements might be implemented in hardware, software (including portable software, such as applets), or both. Further, connection to other computing devices such as network input/output devices may be employed.

Storage media and computer readable media for containing code, or portions of code, can include any appropriate media known or used in the art, including storage media and communication media, such as, volatile and non-volatile, removable and non-removable media implemented in any method or technology for storage and/or transmission of information such as computer readable instructions, data structures, program modules, or other data, including RAM, ROM, Electrically Erasable Programmable Read-Only Memory (EEPROM), flash memory or other memory technology, Compact Disc Read-Only Memory (CD-ROM), digital versatile disk (DVD), or other optical storage, magnetic cassettes, magnetic tape, magnetic disk storage, or other magnetic storage devices, or any other medium which can be used to store the desired information and which can be accessed by the system device. Based on the disclosure and teachings provided, a person of ordinary skill in the art will appreciate other ways and/or methods to implement the various embodiments.

The specification and drawings are, accordingly, to be regarded in an illustrative rather than a restrictive sense. However, it will be evident that various modifications and changes may be made thereunto without departing from the broader spirit and scope of the invention as set forth in the claims. Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a,” “an,” “the,” and similar referents in the context of describing the disclosed embodiments (especially in the context of the following claims) are to be construed to cover both the singular and the plural, unless otherwise indicated or clearly contradicted by context. The terms “comprising,” “having,” “including,” and “containing” are to be construed as open-ended terms (i.e., meaning “including, but not limited to,”) unless otherwise noted. The term “connected,” where unmodified and referring to physical connections, is to be construed as partly or wholly contained within, attached to, or joined together, even if there is something intervening. Recitation of ranges of values are merely intended to serve as a shorthand method of referring individually to each separate value falling within the range, unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal. The use of the phrase “based on,” unless otherwise explicitly stated or clear from context, means “based at least in part on” and is not limited to “based solely on.”

Conjunctive language, such as phrases of the form “at least one of A, B, and C,” or “at least one of A, B and C,” is understood with the context as used in general to present that an item, term, etc., may be either A or B or C, or any nonempty subset of the set of A and B and C, unless specifically stated otherwise or otherwise clearly contradicted by context. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

Operations of processes described can be performed in any suitable order unless otherwise indicated or otherwise clearly contradicted by context. Processes described (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising instructions executable by one or more processors. The computer-readable storage medium may be non-transitory. In some embodiments, the code is stored on a set of one or more non-transitory computer-readable storage media having stored thereon executable instructions that, when executed (i.e., as a result of being executed) by one or more processors of a computer system, cause the computer system to perform operations described herein. The set of non-transitory computer-readable storage media may comprise multiple non-transitory computer-readable storage media and one or more of individual non-transitory storage media of the multiple non-transitory computer-readable storage media may lack all of the code while the multiple non-transitory computer-readable storage media collectively store all of the code. Further, in some embodiments, the executable instructions are executed such that different instructions are executed by different processors. As an illustrative example, a non-transitory computer-readable storage medium may store instructions. A main CPU may execute some of the instructions and a graphics processor unit may execute other of the instructions. Generally, different components of a computer system may have separate processors and different processors may execute different subsets of the instructions.

Accordingly, in some embodiments, computer systems are configured to implement one or more services that singly or collectively perform operations of processes described herein. Such computer systems may, for instance, be configured with applicable hardware and/or software that enable the performance of the operations. Further, computer systems that implement various embodiments of the present disclosure may, in some embodiments, be single devices and, in other embodiments, be distributed computer systems comprising multiple devices that operate differently such that the distributed computer system performs the operations described and such that a single device may not perform all operations.

The use of any examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, although above-described elements may be described in the context of certain embodiments of the specification, unless stated otherwise or otherwise clear from context, these elements are not mutually exclusive to only those embodiments in which they are described; any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

What is claimed is:

1. A system, comprising:
at least one processor; and
a memory comprising instructions that, in response to execution by the at least one processor, cause the system to at least:
maintain a diagnostic model of a patient, the diagnostic model adapted based at least in part on a condition of the patient;
receive information usable as input to the diagnostic model, the information obtained from a monitoring device located in an environment of the patient; and
generate a recommendation of at least one of diagnosis or treatment, the recommendation based at least in part on output of the diagnostic model, the output based at least in part on the input obtained from the monitoring device.

2. The system of claim 1, wherein the information received from the monitoring device is screened, based at least in part on a second diagnostic model, for relevance to at least one of diagnosis or treatment.

3. The system of claim 1, the memory comprising instructions that, in response to execution by the at least one processor, cause the system to at least:
track movement of the patient observed by the monitoring device;
compare the tracked movement to movements predicted based at least in part on a skeletal model and information indicative of a rehabilitative exercise; and
generate the recommendation based at least in part on the comparison.

4. The system of claim 1, the memory comprising instructions that, in response to execution by the at least one processor, cause the system to at least:
receive information, obtained by the monitoring device, indicative of compliance with a treatment regime, wherein sharing of the information by the monitoring device is authorized by the patient.

5. The system of claim 1, wherein the recommendation comprises information indicative of a diagnosis of at least one of an injury or illness.

6. The system of claim 5, the memory comprising instructions that, in response to execution by the at least one processor, cause the system to at least:
initiate a workflow that, upon execution, schedules a follow-up action in response to the diagnosis.

7. A computer-implemented method, comprising:
maintaining a diagnostic model of a patient, the diagnostic model adapted based at least in part on a condition of the patient;
receiving information usable as input to the diagnostic model, the information obtained from a monitoring device located in an environment of the patient; and
generating a recommendation of at least one of diagnosis or treatment, the recommendation based at least in part on output of the diagnostic model, the output based at least in part on the input obtained from the monitoring device.

8. The computer-implemented method of claim 7, wherein the condition to which the diagnostic model is adapted comprises at least one of physiological data or anatomical data particular to the patient.

9. The computer-implemented method of claim 7, wherein the diagnostic model simulates a physical or anatomical function.

10. The computer-implemented method of claim 7, further comprising:
screening information collected from a monitoring device for authorization to use the information, based at least in part on a second diagnostic model.

11. The computer-implemented method of claim 10, wherein the second diagnostic model determines relevance of the information to a medical condition for which monitoring is authorized by a user of the monitoring device.

12. The computer-implemented method of claim 7, wherein the recommendation comprises information indicative of performance of a rehabilitative exercise.

13. The computer-implemented method of claim 12, further comprising:
tracking movement of the patient observed by the monitoring device;
comparing the tracked movement to movements predicted based at least in part on a skeletal model and information indicative of a rehabilitative exercise; and
generating the recommendation based at least in part on the comparing.

14. The computer-implemented method of claim 7, further comprising:
providing a reminder to assist in compliance with a treatment regime, based at least in part on information indicative of compliance received from the monitoring device, wherein sharing of the information by the monitoring device is authorized by the patient.

15. The computer-implemented method of claim 14, further comprising:
obtaining the information indicative of compliance by recognizing, in video data obtained from the monitoring device, an image of a container of medication.

16. A non-transitory computer-readable storage medium having stored thereon instructions that, in response to execution by at least one processor of a computer, cause the computer to at least:
maintain a diagnostic model of a patient;
receive visual information obtained by a camera of a monitoring device;
convert the visual information to a format usable as input to the diagnostic model; and
obtain a recommendation of at least one of diagnosis or treatment, the recommendation based at least in part on output of the diagnostic model.

17. The non-transitory computer-readable storage medium of claim 16, wherein conversion of the visual information to the format usable as input to the diagnostic model is based at least in part on a skeletal model of the patient.

18. The non-transitory computer-readable storage medium of claim 16, wherein the recommendation comprises instructions for performing a rehabilitative exercise, the instructions displayed in a visual format by the monitoring device.

19. The non-transitory computer-readable storage medium of claim 16, having stored thereon instructions that, in response to execution by at least one processor of a computer, cause the computer to at least:
detect, in visual information obtained by the monitoring device, information indicative of compliance with a medication regime, the visual information authorized for use in detecting compliance; and
generate the recommendation based at least in part on the information indicative of compliance with the medication regime.

20. The non-transitory computer-readable storage medium of claim 16, having stored thereon instructions that, in response to execution by at least one processor of a computer, cause the computer to at least:
track movement of the patient observed by the monitoring device;
compare the tracked movement to movements predicted based at least in part on a skeletal model and information indicative of a rehabilitative exercise; and
generate the recommendation based at least in part on the comparison.
